(19) United States
(12) Patent Application Publication
(10) Pub. No.: US 2002/0111563 A1
(43) Pub. Date: Aug. 15, 2002

(54) Vacuum skin sample collection apparatus

(76) Inventor: Stanley Hall, Ripon, CA (US)

Correspondence Address: ROBERT S. SMITH, LAW OFFICES OF ROBERT S. SMITH, SUITE 306, 1131-O TOLLAND TURNPIKE, MANCHESTER, CT, 06040, US

(21) Appl. No.: 09/782,654

(22) Filed: Feb. 13, 2001

(51) Int. Cl. A61B 10/00

(52) U.S. Cl. 600/566

(57) ABSTRACT

Apparatus for collection of a skin sample from a patient that includes an elongated generally tubular barrel having first and second axial extremities and having an interior chamber in communication with the first axial extremity. The first axial extremity of the generally tubular barrel has a sharp edge. The apparatus further includes apparatus for connecting the interior chamber to an associated vacuum source and apparatus for directing a vacuum from the associated vacuum source to the sharp edge. Some forms of the invention include a stop member disposed within the barrel for limiting the thickness of the skin sample. The stop member may be a perforate body. The apparatus may further include apparatus for securing the stop member in a plurality of discrete axial positions whereby the thickness of the skin sample may be defined before the collection process starts. The apparatus may include a visual display apparatus indicating the thickness of the skin sample for each of a plurality of axial positions of the stop member within the barrel.

### BACKGROUND OF THE INVENTION

The invention relates to medical apparatus and particularly to apparatus for collecting skin specimens or samples suitable for performing a biopsy as well as other medical procedures. While the description herein may refer primarily to apparatus for obtaining a sample for a biopsy, those skilled in the art will recognize that it also has application for obtaining other specimens suitable for other medical testing. In addition the apparatus in accordance with the present invention is also useful in applications where skin plug samples are needed. For example, hair plugs may be extracted with the apparatus in accordance with present invention from a patient for placement in a different part of the scalp of the donor or to a different recipient. In some cases the apparatus of the present invention may be used to extract a skin plug as a prelude to implanting a hair plug.

The prior art apparatus employs a skin plug cutter unit that includes a small diameter tube with a sharpened end. The sharp end is pushed downward into the skin and twisted. This pushing, twisting and cutting action helps the cutting process. The end of the chamber has to be round so that rotation of the tube will cut the surface of the skin with a minimum of trauma to the specimen being collected as well as the surrounding tissue. Thereafter the skin plug produced in this manner is mechanically lifted, cut with a scalpel or scissors and saved in a vessel with a protective liquid. The skin plug is then inspected and tested.

A primary problem with this and other prior art methods is the damage done to the skin plug during the removal process. The living cells in the plug are generally crushed, squeezed of fluid, and otherwise damaged by mechanical extraction with tweezers or a needle. This makes testing very difficult and leads to test results that are not reliable. In addition the round skin plug cutout is slow to heal as a circle has a larger minimum dimension for a given area, as compared to an oval shape.

### SUMMARY OF THE INVENTION

It is an object of the invention to provide an apparatus to collect skin plugs for medical procedures and which does not damage the skin sample.

Another object invention is to provide apparatus that is practical and which enables a technician to easily collect a skin sample including skin plug samples.

It has now been found that these and other objects of the invention may be attained in apparatus for collection of a skin sample that includes an elongated generally tubular barrel having first and second axial extremities and having an interior chamber in communication with the first axial extremity. The first axial extremity of the generally tubular barrel has a sharp edge. The apparatus further includes means for connecting the interior chamber to an associated vacuum source and means for directing a vacuum from the associated vacuum source to the sharp edge.

Some forms of the invention include a stop member disposed within the barrel for limiting the thickness of the skin sample. The stop member may be a perforate body. The apparatus may further include means for securing the stop member in a plurality of discrete axial positions whereby the thickness of the skin sample may be defined before the collection process starts. The apparatus may include a visual display means indicating the thickness of the skin sample for each of a plurality of axial positions of the stop member within the barrel.

The apparatus may further include a regulator for regulating the vacuum proximate to the sharp edge. The apparatus may also further include a valve for selectively connecting the interior chamber to ambient and to selectively reduce the vacuum within the interior chamber. The cutting edge may be circular, oval, or various other shapes.

### BRIEF DESCRIPTION OF THE DRAWING

The invention will be better understood by reference to the accompanying drawing in which:

FIG. 1 is an enlarged partially cross-section elevational schematic view of a preferred form of the invention.

FIG. 2a is a partial cross-section elevational schematic view of apparatus similar to that illustrated in FIG. 1 as the apparatus is first pressed against the skin of a patient as part of the process of collecting a skin specimen.

FIG. 2b is a partial cross-section elevational schematic view of the apparatus illustrated in FIG. 2a as a vacuum is first applied to the skin of the patient while the apparatus is pressed against the skin of the patient.

FIG. 2c is a partial cross-section elevational schematic view of the apparatus illustrated in FIG. 2a as the apparatus is pushed further into the skin of the patient concurrently with the application of a vacuum to the surface of the skin.

FIG. 2d is a partial cross section elevational schematic view of the apparatus illustrated in FIG. 2a after the apparatus has been pushed still further into the surface of the skin of the patient and the outer surface of the skin is disposed in abutting relationship with a stop that is adjustable to define the thickness of the specimen.

FIG. 2e is a partial cross-section elevational schematic view of the apparatus illustrated in FIG. 2a after the skin plug or specimen has been drawn into the barrel of the apparatus until the specimen reaches the stop that determines the thickness of the specimen and after the apparatus has been withdrawn from the surface of the skin of the patient with the aid of a scalpel.

FIG. 2f is a partial cross-section elevational schematic view of the apparatus illustrated in FIG. 2a after the apparatus has been moved away from the surface of the skin of the patient with the skin sample tissue plug, that has been removed from the patient's surrounding tissue, is retained within the apparatus.

FIG. 2g is a partial cross-section elevational schematic of the apparatus illustrated in FIG. 2a that illustrates the sample tissue plug being ejected into a sample retention vial.

FIG. 3 is a partial cross-section elevational schematic of the apparatus illustrated in FIG. 2a that illustrates view of one embodiment of the apparatus that includes a tissue or plug stop, an adjustment that enables the user to predefine a desired specimen or sample thickness with an indicator to visually display the specimen thickness.

FIG. 3a is an elevation view of another similar indicator used to visually display the specimen thickness.

FIG. 4 is a schematic view of the apparatus illustrated in FIG. 1 together with a vacuum connection, control valve, vacuum gauge, and vacuum regulator.

FIG. 5 is a partial schematic view of apparatus similar to the apparatus illustrated in FIG. 1 that has an oval shaped cutting edge surface that engages the surface of the patients skin to produce an oval shaped plug.

FIG. 6 is a partial cross-section schematic view of the apparatus FIG. 1 that illustrates a screw type adjustment for actually positioning the skin plunger assembly stop.

FIGS. 7a, 7b, 7c, 7d, and 7e, are cross-section schematic views of various forms of a screw type adjustment skin plunger assembly for the apparatus illustrated in FIG. 1.

FIG. 8 is a cross-section schematic view of apparatus similar to the illustrated in FIG. 1 that incorporates a screw type adjustment skin plunger assembly.

### DESCRIPTION OF THE PREFERRED EMBODIMENTS

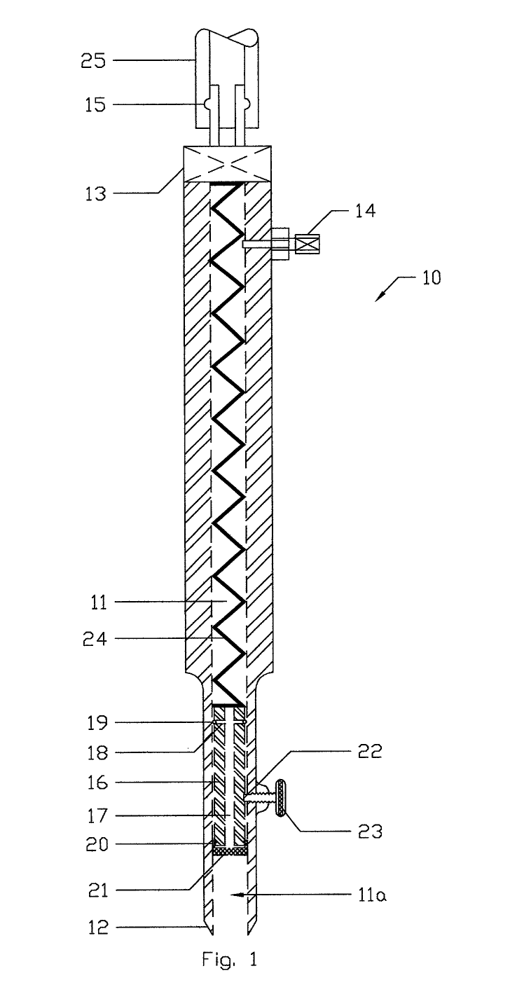

Referring now to FIG. 1 there is shown a vacuum skin sample collection apparatus 10 that includes a barrel or vacuum chamber cylinder 11. The lowest (as viewed) axial extremity of the barrel 11 is a sharp cutting edge 12. In the embodiment illustrated in FIG. 1 the cutting edge 12 is circular although other embodiment may have other contours. Disposed at the upper (as viewed) axial extremity of the barrel 11 is a vacuum pressure regulator for control valve 13. A vacuum release valve 14 extends through the sidewall of the barrel 11. The vacuum release valve 14 enables the user to vent the air to ambient and from the barrel 11 to reduce the vacuum within the barrel 10 to avoid an excess vacuum at the time of collection of the specimen. In addition the operator of the apparatus will also use the vacuum release valve 14 to release the specimen from the vacuum skin sample collection apparatus 10 into a collection vial.

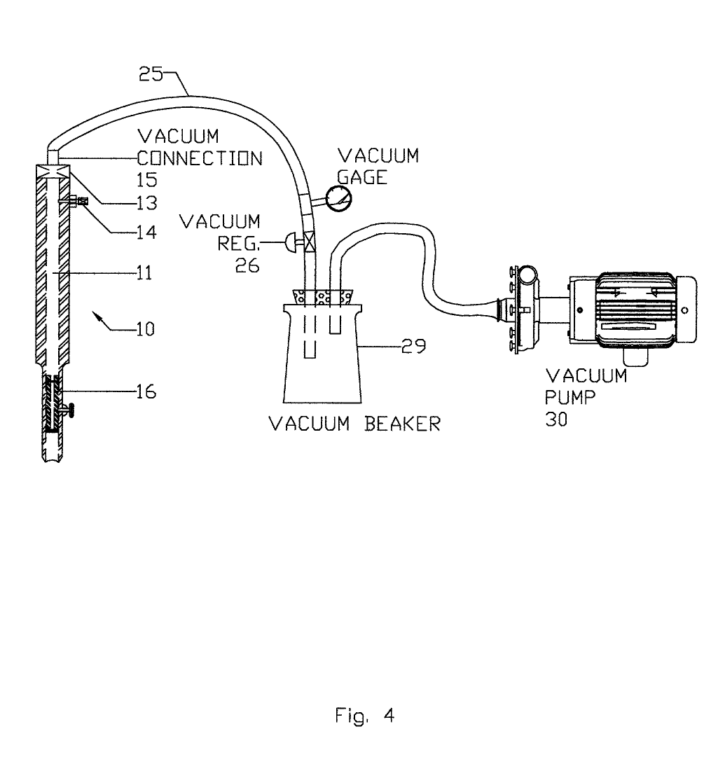

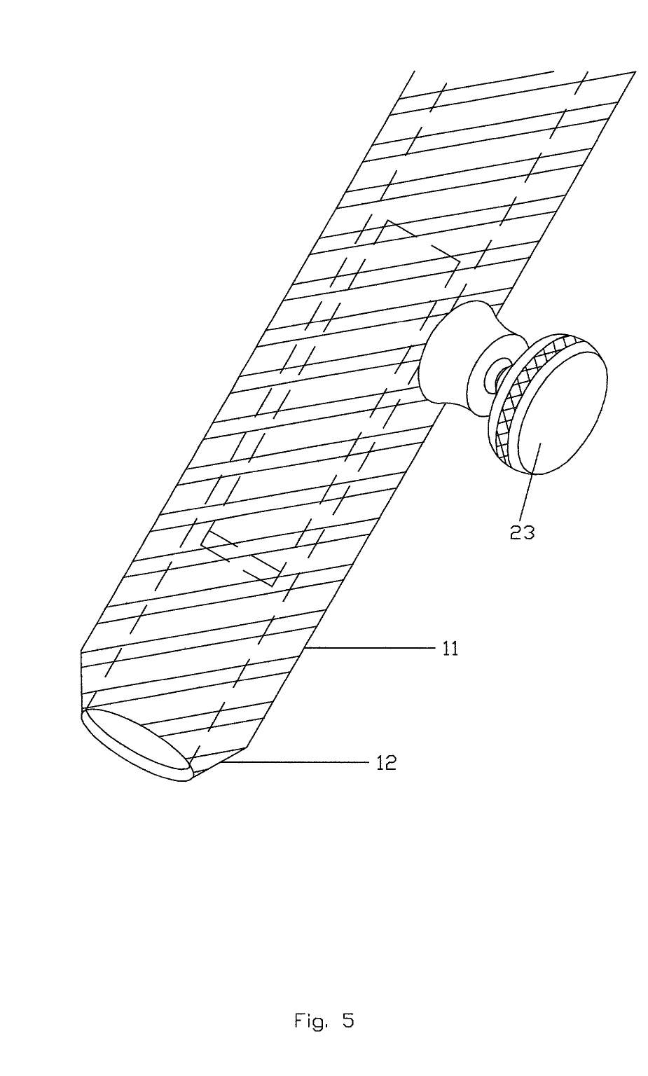

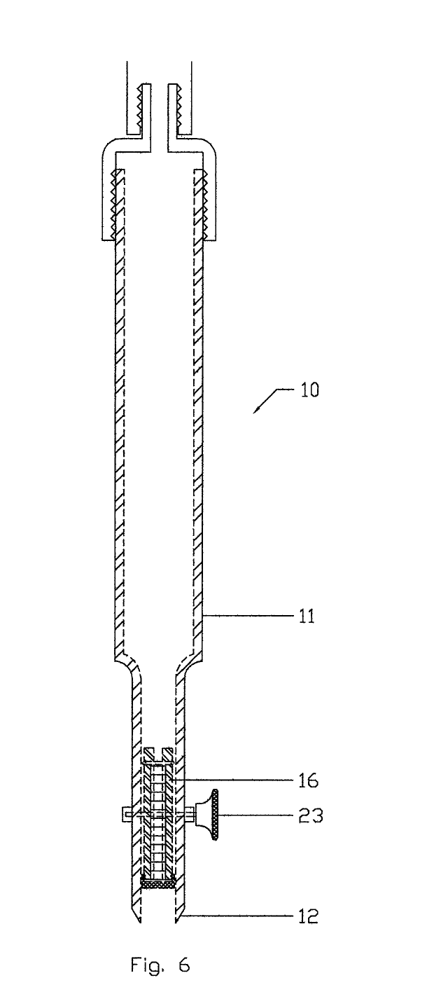

Also disposed at the upper axial extremity of the barrel 11 is a vacuum tube connector 15 for connection to a vacuum pump 30 (shown in FIG. 4) that produces the vacuum within the barrel 11 that assists in achieving withdrawal of a skin plug from a patient. The vacuum skin sample collection apparatus 10 further includes a cylinder assembly 16 disposed at the lower (as viewed) axial extremity of the barrel 11. The cylinder assembly 16 has a central passageway 17 that allows passage of air being withdrawn by a vacuum pump (shown in FIG. 4) through the vacuum tube connector 15. An O-ring seal 19 is disposed within the groove 18 proximate to the upper (as viewed) axial extent of the cylinder assembly 16. The seal 19 insures that the vacuum drawn by the vacuum pump 30 and connected to the vacuum tube connector 15 is directed to the central passageway 17.

Attached to the lower (as viewed) axial extremity of the cylinder assembly 16 is a plug stop 21. The plug stop 21 is a perforate element intended to limit the maximum travel of a skin plug into the barrel 11 of the vacuum skin collection apparatus 10. The cylinder assembly 16 is provided with a cylinder stop 22 and is engaged by an adjustment screw 23 that extends through the wall of the barrel 11 to abut the cylinder stop 22. More specifically, the adjustment screw 23 engages the cylinder stop 22 to lock the cylinder assembly 16 with the associated plug stop 21 in an axial position with respect to the barrel 11 corresponding to a desired skin plug thickness. Disposed within the barrel 11 is a spring 24 that biases the axial position of the cylinder assembly 16 with respect to the axial extent of the barrel 11.

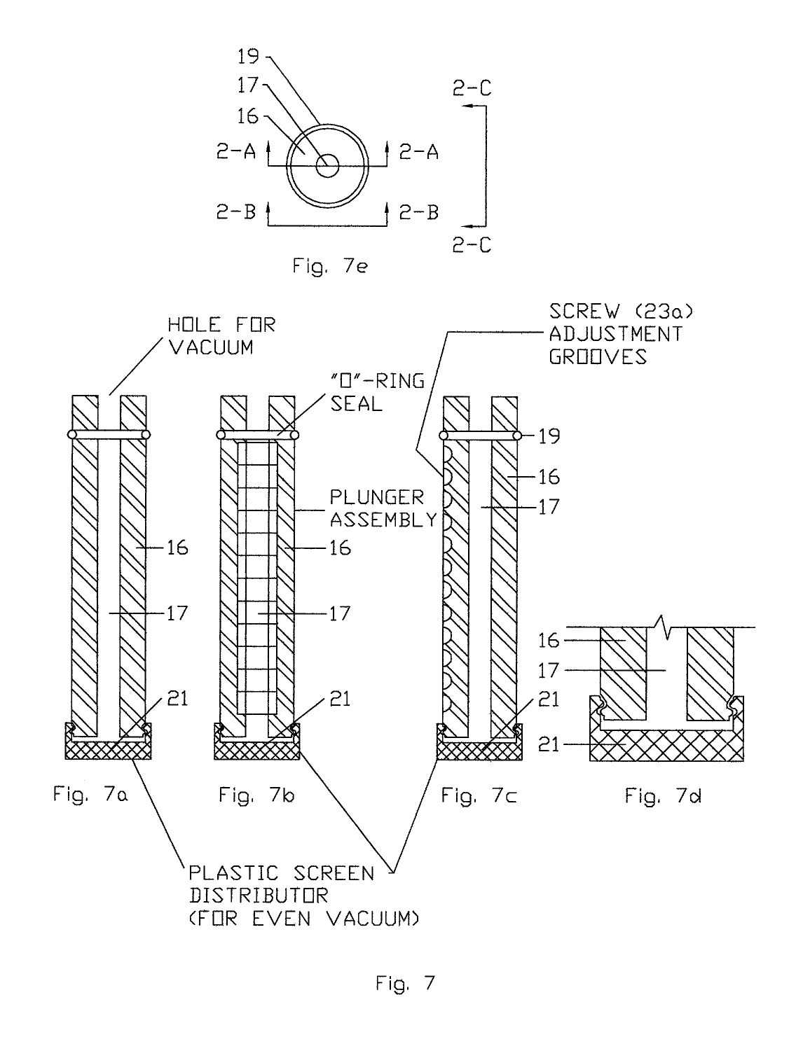

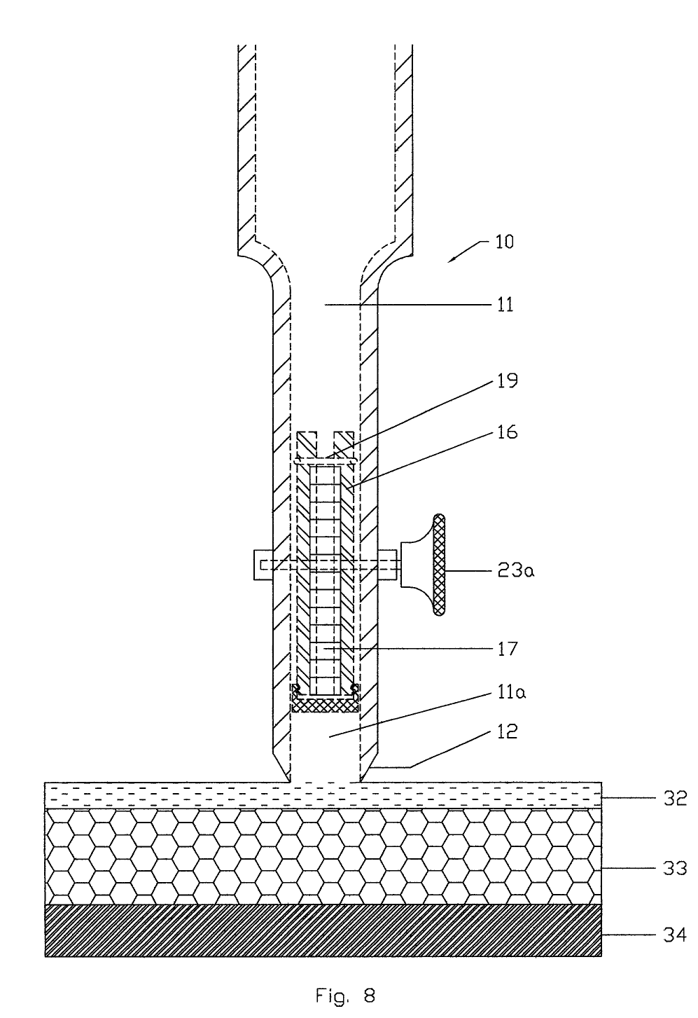

Further details of the construction of the cylinder assembly are shown in FIGS. 7a-7d. These views illustrate the connection between the plug stop 21 and the rest of the cylinder assembly 16. FIG. 7c illustrates a plurality of recesses along the side of the cylinder assembly that are dimensioned and configured for receiving the adjustment screw 23 to lock the cylinder assembly in place.

Connected to the vacuum tube connector 15 is a vacuum hose 25. As best seen in FIG. 4 a vacuum regulator 26 and a vacuum gauge are disposed within the vacuum hose 25. The vacuum hose 25 also connects to a vacuum beaker 27.

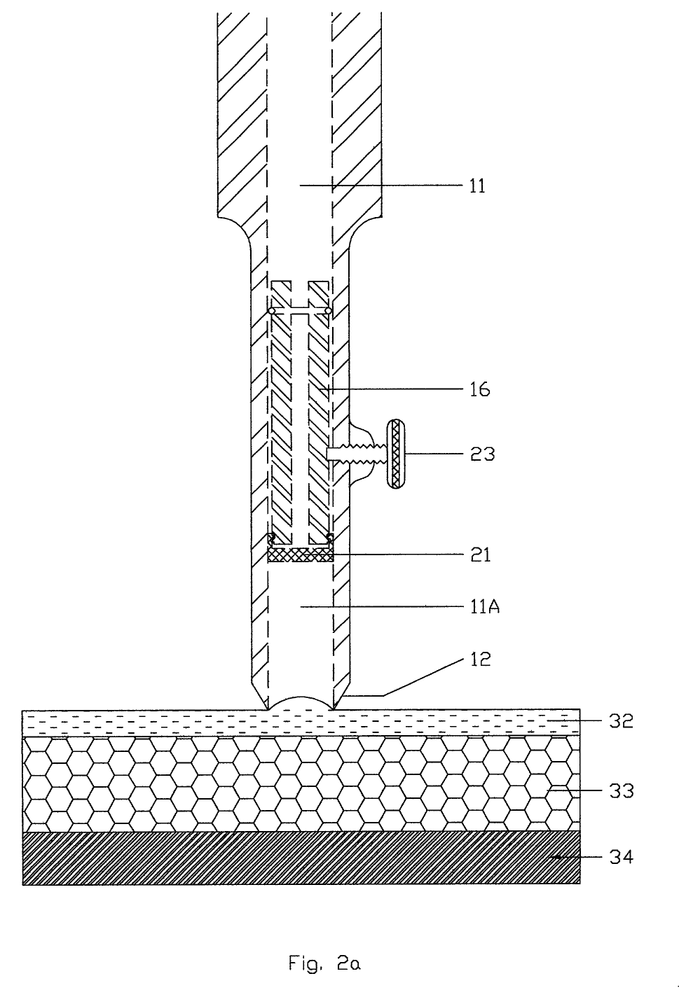

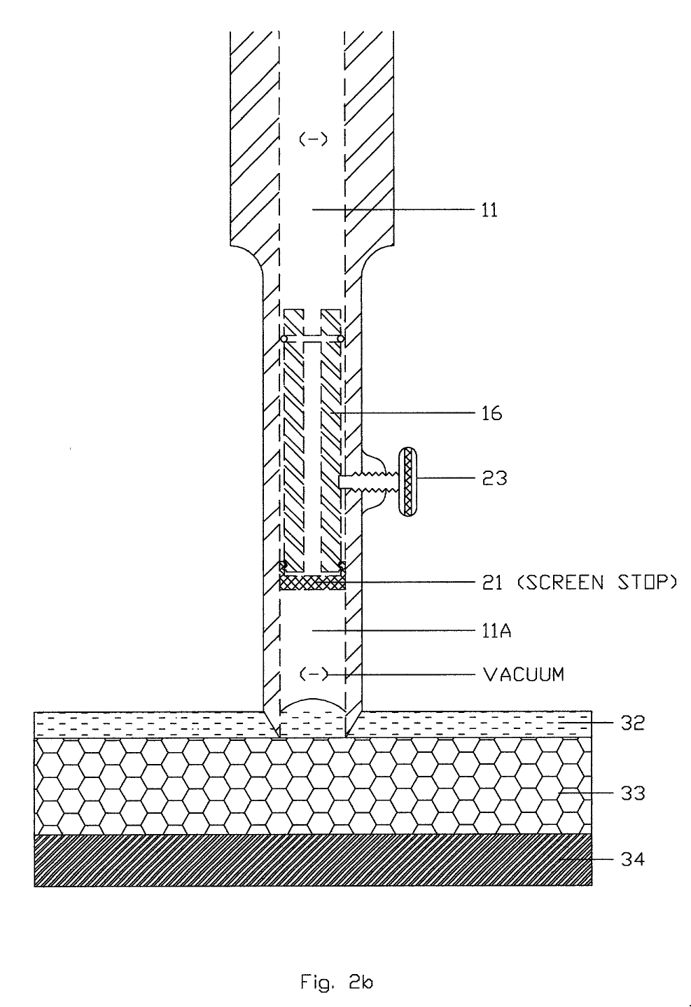

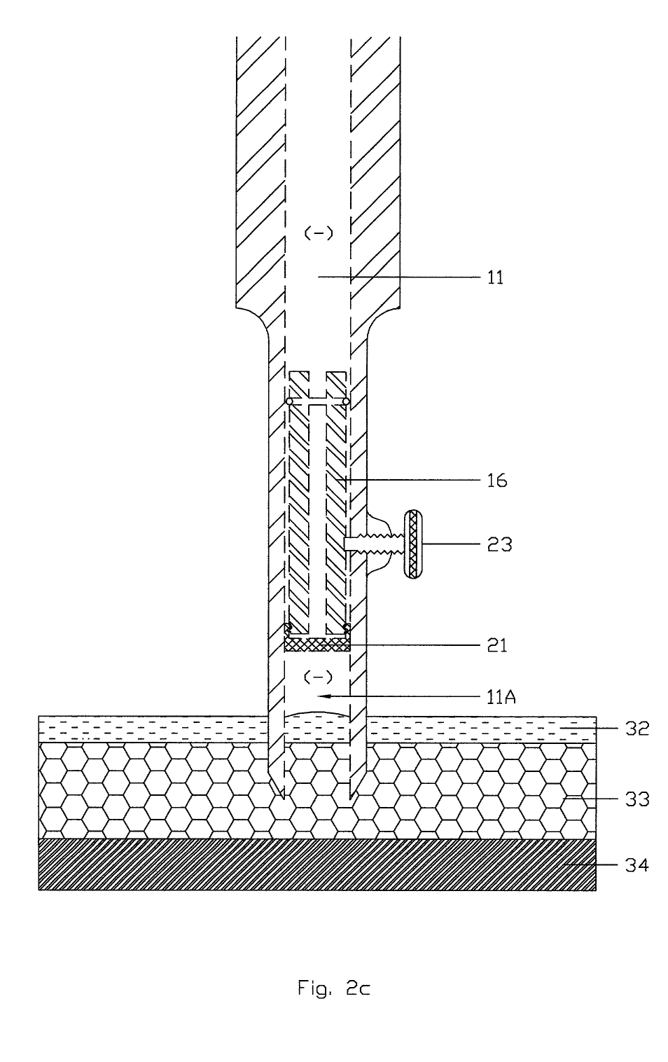

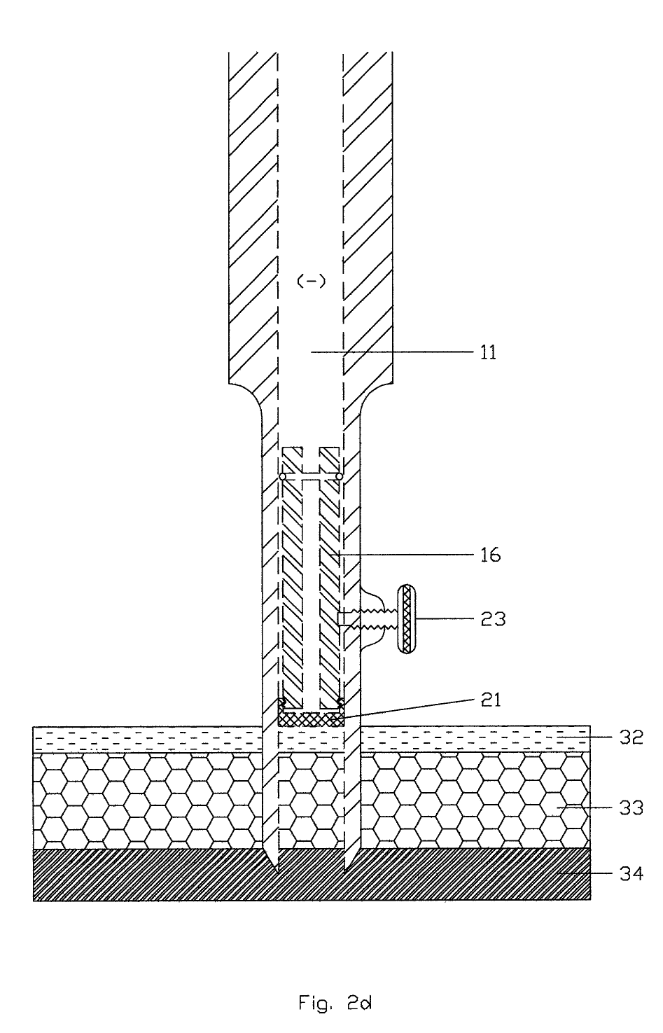

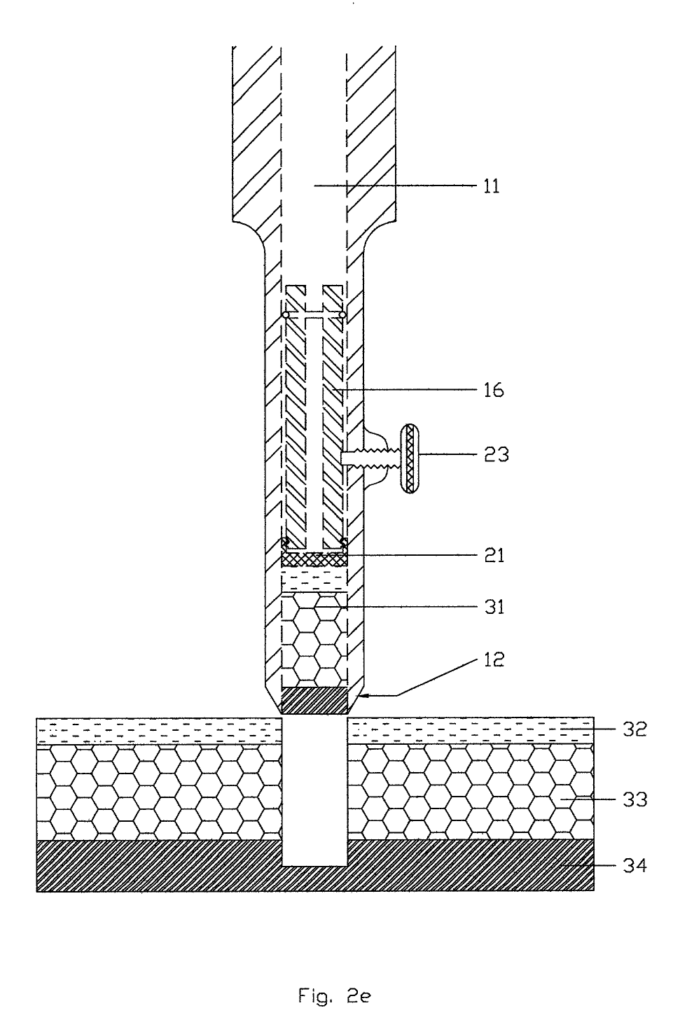

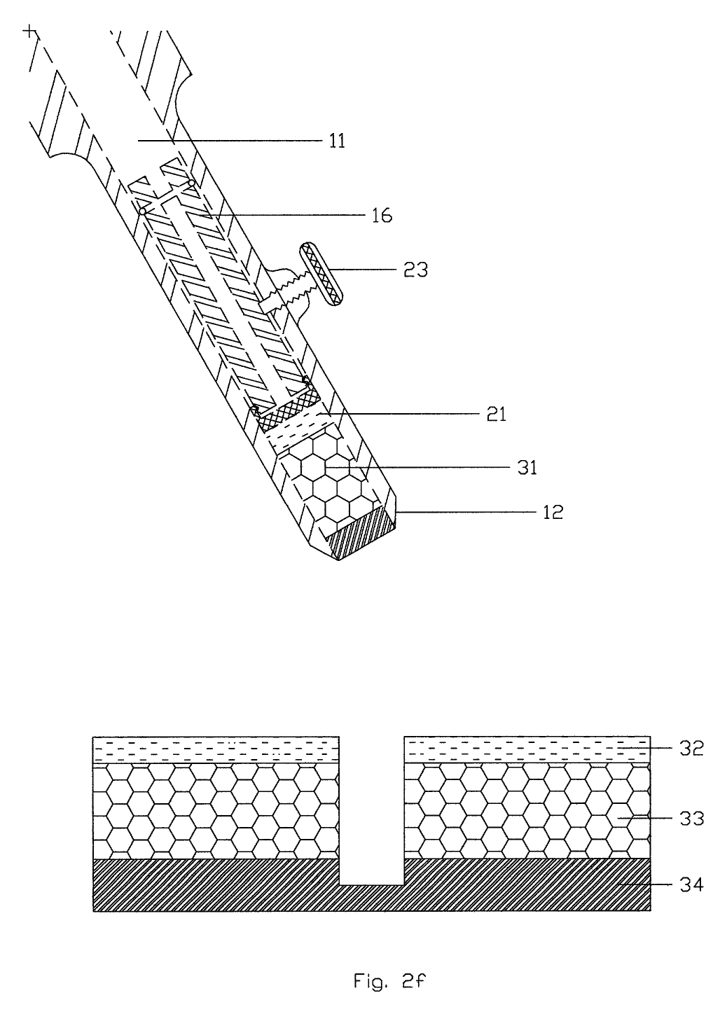

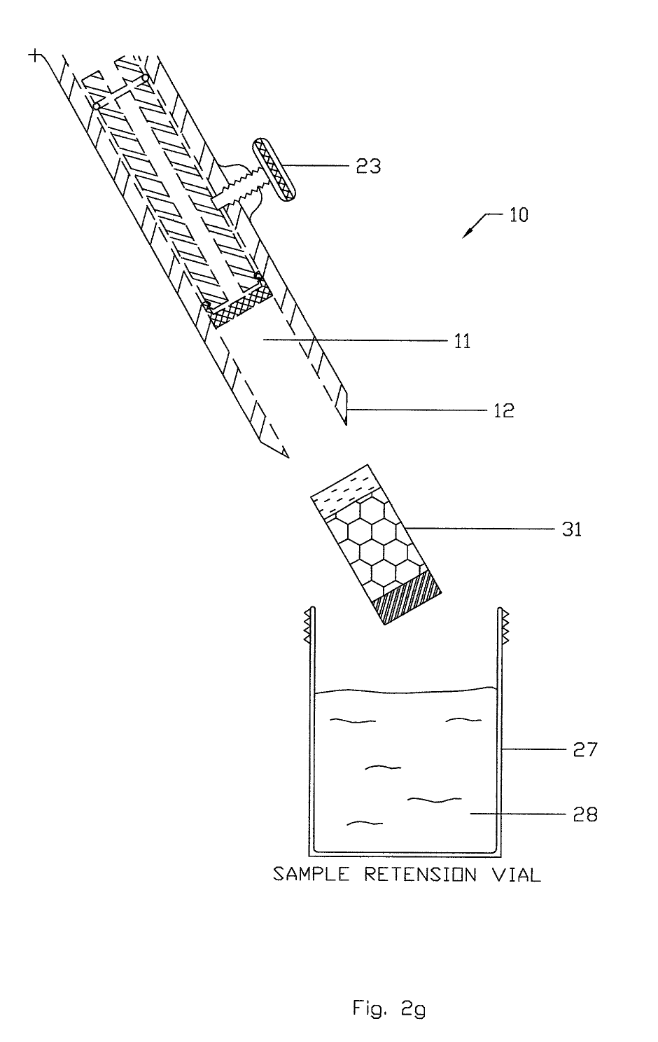

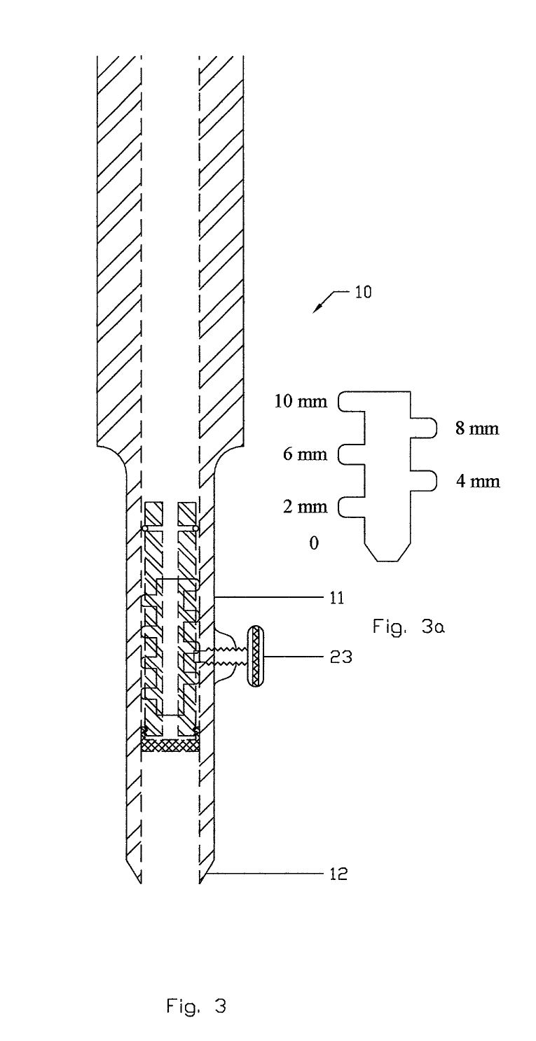

FIGS. 2a-2g illustrate the sequential steps involved in withdrawing a skin plug from the surrounding skin tissue of the patient. FIG. 2a shows the apparatus first being pressed against the skin of a patient as part of the process of collecting specimen a skin specimen. FIG. 2b illustrates the relationship as a vacuum is first applied to the skin of the patient while the apparatus is pressed against the skin of the patient and the cutting edge 12 has penetrated the epidermis 32. FIG. 2c is a view of apparatus illustrated in FIG. 2a as the apparatus is pushed further into the skin of the patient concurrently with the application of a vacuum to the surface of the skin. More specifically, the cutting edge 12 has passed completely through the epidermis 32 and entered the dermis 33 in this step. FIG. 2d shows the apparatus after it has been pushed still further into the surface of the skin of the patient and the outer surface of the skin is disposed in abutting relationship with the stop 21 that is adjustable to define the thickness of the specimen. More specifically, because of the setting of the plug stop 21 the cutting edge 12 has passed completely through the epidermis 32, the dermis 33 and part of subcutaneous fat layer 34. FIG. 2e shows the apparatus and skin after the skin plug or specimen has been drawn into the barrel of the apparatus until the specimen reaches the stop that determines the thickness of the specimen and after the apparatus has been withdrawn from the surface of the skin of the patient with the aid of a scalpel. FIG. 2f shows the apparatus and skin after the apparatus has been moved away from the surface of the skin of the patient with the skin sample tissue plug. The plug has been removed from the patient's surrounding tissue and is retained within the apparatus. FIG. 2g is a schematic of the sample tissue plug being ejected into a sample retention vial 27 that holds a protective fluid 28. The project fluid 28 is provided to maintain the integrity of the specimen.

This invention utilizes a vacuum to assist in the extraction of a specimen from the surrounding tissue of the patient. Stated another way, the apparatus 10 in accordance with the preferred form of the invention utilizes a vacuum to position a skin plug within a receiving chamber within the barrel 11. After the vacuum draws the skin plug into the journal of the vacuum skin sample collection apparatus 10, the plug is severed from the surrounding tissue by a scalpel. The skin plug travels up the chamber to a preset stop 21. The stop 21 is preset to a position corresponding to a specific plug thickness with the aid of a visual indicator. Thus, the thickness is predefined by the vacuum skin sample collection apparatus 10. The More specifically, the cylinder assembly 16 is movable axially within the barrel 11 to position the stop 21. Thus, the thickness of the plug is known prior to severing the plug from the surrounding tissue. The skin test plug is severed with a sharp scalpel or other cutting blade. The skin plug is then deposited into a sample vessel 27 containing a storage solution 28. The vacuum pressure is controlled throughout the procedure to minimize any damage to the skin plug.

In some embodiments of the invention of the skin specimen is a cylindrical plug having a circular cross-section. In other embodiments of the invention of plug has an oval shaped cross-section. An oval cross-section is advantageous because the recess remaining after removal of the plug is much easier to close and may even be closed with a butterfly bandage, If a suture is required, a single suture will be effective in many cases. It will be understood that an opening having an oval shape and having the same cross-section area as a round plug will be easier to close and will heal faster.

The vacuum skin sample collection apparatus in accordance with the present invention includes a cylindrical or oval cylinder with an adjustable stop feature. An adjustment screw controls the stop feature. This allows for presetting the skin plug to any thickness desired. To minimize the need for twisting and rocking action of prior art units this invention includes a connection to the chamber for a vacuum line. A control valve and vacuum regulator allows for the proper vacuum desired. The regulator allows for different vacuum settings for different skin types. As the end of the chamber is lightly placed against the skin a vacuum is produced within the chamber. The skin within and in contact with the end of the vacuum skin sample collection apparatus 10 is gently drawn into the chamber due to the vacuum action. The skin plug travels up the chamber until it reaches a preset stop. This stop has a small opening for the evacuated air between the stop and the skin to travel. As the air is evacuated the skin touches the stop. The vacuum may be partly reduced at this contact point by means of a by-pass opening to ambient air to avoid excess vacuum on the skin plug. The skin plug within the chamber is then lifted to skin level, severed and deposited in a sample vessel that contains a protective fluid.

By providing a vacuum to draw the skin into a chamber the chamber may be of any desired shape. The ideal skin plug shape is an oval as this allows for the edges of the skin to come together by use of tape, avoiding sutures, and reduce the risk of scaring.

Advantageously, the vacuum skin sample collection apparatus in accordance with present invention achieves the collection of skin plug samples that are of uniform size, shape, thickness and free of the crushed or squeezed cells as with conventional samples. The testing laboratory will receive the samples in an ideal condition to obtain reliable biopsy test results. Advantageously, the vacuum skin sample collection apparatus enables the collection of skin samples in a manner that the patient will find to be less painful. In addition the collection process where the apparatus of present invention will cause less trauma to the surrounding tissue. Accordingly, the surrounding tissue will heal faster. Because the apparatus in accordance with present invention enables collection of an oval plug, the edges of the skin can drawn together in many cases with tape. Accordingly, healing occurs more rapidly, the danger of infection is reduced and the possibility of scarring is reduced.

Although the vacuum skin sample collection apparatus has been shown in forms capable of collecting samples that are circular as well as samples that are oval, those skills in the art will recognize that other forms of present invention may have many other alternative shapes and sizes.

The vacuum skin sample collection apparatus in accordance with present invention is preferably manufactured of stainless steel. Ordinarily the barrel or cylinder of the apparatus is manufactured from stainless steel cylinder tubing. The complete apparatus includes an adjustable stop and a vacuum connector as well as adjustment valves, a regulator and a vacuum gauge are part of the complete system. The materials used in the construction may include an FDA approved plastic that can be disposed of after a single use.

While the present invention has been described with reference to the preferred embodiments illustrated in the drawing, the detailed description thereof is not intended to limit the scope of the invention as claimed in the appended claims.

I claim:

1. Apparatus for collection of a skin sample which comprises:
an elongated generally tubular barrel having first and second axial extremities and having an interior chamber in communication with said first axial extremity, said first axial extremity of said generally tubular barrel having a sharp edge; and
means for connecting said interior chamber to an associated vacuum source and means for directing a vacuum from the associated vacuum source to said sharp edge.

2. The apparatus in accordance with claim 1 further including a stop member disposed within said barrel for limiting the thickness of the skin sample.

3. The apparatus in accordance with claim 2 wherein said stop member is a perforate body.

4. The apparatus in accordance with claim 3 further including means for securing said stop member in a plurality of discrete axial positions whereby the thickness of the skin sample may be defined before the collection process starts.

5. The apparatus in accordance with claim 4 wherein said apparatus includes a visual display means indicating the thickness of the skin sample for each of a plurality of axial positions of said stop member within said barrel.

6. The apparatus in accordance with claim 5 further including a regulator for regulating the vacuum proximate to said sharp edge.

7. The apparatus in accordance with claim 6 further including a valve for selectively connecting said interior chamber to ambient and to selectively reduce the vacuum within said interior chamber.

8. The apparatus in accordance with claim 7 wherein said cutting edge is circular.

9. The apparatus in accordance with claim 7 wherein said cutting edge is oval.

10. The apparatus in accordance with claim 7 wherein said barrel has a circular cross-section.
